(19) United States
(12) Patent Application Publication
(10) Pub. No.: US 2024/0331848 A1
(43) Pub. Date: Oct. 3, 2024

(54) METHOD AND APPARATUS FOR QUANTITATIVE CHRONIC OBSTRUCTIVE PULMONARY DISEASE EVALUATION USING ANALYSIS OF EMPHYSEMA

(71) Applicants: CORELINE SOFT CO., LTD., Seoul (KR); The Asan Foundation, Seoul (KR); UNIV OF ULSAN FOUNDATION FOR INDUSTRY COOPERATION, Ulsan (KR)

(72) Inventors: Joon Beom SEO, Seoul (KR); Sang Min LEE, Seoul (KR); Jihye YUN, Seoul (KR); Seungbin BAE, Goyang-si (KR); Donghoon YU, Gimpo-si (KR); Jaeyoun YI, Seoul (KR)

(21) Appl. No.: 18/621,438

(22) Filed: Mar. 29, 2024

(30) Foreign Application Priority Data
Mar. 31, 2023 (KR) 10-2023-0043179

(51) Int. Cl. G16H 30/40; G06T 7/00

(52) U.S. Cl. CPC G16H 30/40; G06T 7/0012; G06T 2207/30061

(57) ABSTRACT

A method according to an embodiment of the present disclosure includes obtaining a first finding region in a first medical image and size information of the first finding region; identifying a second finding region matching the first finding region in a second medical image obtained after the first medical image; obtaining size information of the second finding region; and generating diagnosis assistance information based on the size information of the first finding region and the second finding region.

### CROSS-REFERENCE TO RELATED APPLICATION(S)

This application claims priority from KR 10-2023-0043179 filed Mar. 31, 2023, which is incorporated herein by reference in its entirety.

### BACKGROUND

### 1. Technical Field

The present invention relates to technology for processing, analyzing, and visualizing medical images, and more particularly to technology for providing the quantitative evaluation of medical images to assist in the diagnosis of Chronic Obstructive Pulmonary Disease (COPD) or emphysema.

### 2. Related Art

The content described in the present section simply provides background information for embodiments and does not constitute prior art.

Unlike X-ray imaging, computed tomography (CT) obtains cross-sectional images of the human body that is cut horizontally. Compared to simple X-ray imaging, CT has the advantage of being able to view structures and lesions more clearly because there are few overlaps between the structures. Furthermore, CT imaging is cheaper and requires a shorter examination time than magnetic resonance imaging (MRI), so that it is a basic examination method when lesions are suspected in most organs and diseases and the detailed examination thereof is required.

Meanwhile, COPD refers to various clinical symptoms in which the alveoli inside the lung are permanently damaged. COPD typically includes emphysema, in which the tissue that constitutes the alveoli is destroyed and cannot function normally. Recently, to quantitatively diagnose emphysema, there has emerged a technology that calculates the contrast of CT images using software and automatically/semi-automatically analyzes regions having a specific contrast value or less as an emphysema area.

To evaluate the degree of progression of COPD or emphysema, it is necessary to compare and read medical images over several months or years.

In a conventional technology, the degree of progression of COPD or emphysema is evaluated by comparing individually obtained quantitative evaluation results or by visually comparing the latest image and the previous image through the naked eyes of a radiologist.

As a result, there are problems in that variations may occur depending on medical image modality, the change in the imaging method, the conditions of a patient during imaging, and the conditions of a radiologist during reading, and/or the like and missing information occurs.

### SUMMARY

The present invention has been conceived to overcome the above-described problems, and one of the objects of the present invention is to provide a consistent index for quantitative analysis using the registration between the latest image and the previous image.

One of the objects of the present invention is to propose an evaluation index, the visualization of the index, and evaluation criteria for the quantitative evaluation of the degree of progression of COPD or emphysema.

One of the objects of the present invention is to provide technology for generating an index that is used to quantitatively evaluate the degree of progression of a specific disease based on changes in the size of a finding region over time by using the follow-up matching of finding regions obtained through thresholding, detection, segmentation, and/or classification in medical images.

According to an aspect of the present invention, there may be provided a method of generating diagnosis assistance information using medical images, the method including: obtaining a first finding region in a first medical image and the size information of the first finding region; identifying a second finding region matching the first finding region in a second medical image obtained after the first medical image; obtaining the size information of the second finding region; and generating diagnosis assistance information based on the size information of the first finding region and the second finding region.

The generating may comprises generating follow-up information including the size information of the first finding region and the second finding region that match each other.

The generating may further comprise generating information about which group the follow-up information corresponds to among a plurality of groups classified based on the size information of the first finding region and the second finding region included in the follow-up information.

The generating may further comprise generating quantitative information for each group related to a distribution of the first finding region or the second finding region to which the follow-up information corresponds for each of the plurality of groups.

The generating quantitative information for each other may comprise generating quantitative information for each group, including at least one of a number of pixels or voxels in finding regions corresponding to each of the plurality of groups, a number of finding regions corresponding to each section, or an area or volume of the finding regions corresponding to each section.

In the method according to the embodiment of the present invention, the size information of the first finding region may be a length of a long axis of the first finding region, and the size information of the second finding region may be a length of a long axis of the second finding region; and the follow-up information may be a change between the length of the long axis of the first finding region and the length of the long axis of the second finding region.

In the method according to the embodiment of the present invention, the size information of the first finding region may be a volume of the first finding region, and the size information of the second finding region may be a volume of the second finding region; and the follow-up information may be a change between the volume of the first finding region and the volume of the second finding region.

The method according to the embodiment of the present invention may further comprise visualizing information about which group the follow-up information corresponds to as the diagnosis assistance information.

The generating may further comprise generating diagnostic assistance information, including information about which section the follow-up information corresponds to among a plurality of sections classified by applying a plurality of thresholds to a change between the size information of the first finding region and the second finding region included in the follow-up information.

The generating may further comprise generating diagnosis assistance information, including quantitative information for each section related to a distribution of first finding region or second finding region to which the follow-up information corresponds for each of the plurality of sections.

In the method according to the embodiment of the present invention, the first finding region may be a region in the first medical image whose intensity is lower than a first threshold; and the second finding region may be a region in the second medical image whose intensity is lower than the first threshold.

In the method according to the embodiment of the present invention, the first finding region may be a finding region related to a respiratory function of a lung in the first medical image; and the second finding region may be a finding region related to the respiratory function of the lung in the second medical image.

According to an embodiment of the present invention, there may be provided an apparatus for generating diagnosis assistance information using medical images, the apparatus comprising: memory configured to store one or more instructions; and a processor configured to execute the one or more instructions; wherein the processor may be further configured to, in accordance with at least one of the instructions: obtain a first finding region in a first medical image and size information of the first finding region; identify a second finding region matching the first finding region in a second medical image obtained after the first medical image; obtain size information of the second finding region; and generate diagnosis assistance information based on the size information of the first finding region and the second finding region.

The processor may be further configured to, when generating the diagnosis assistance information, generate follow-up information including the size information of the first finding region and the second finding region that match each other.

The processor may be further configured to, when generating the diagnosis assistance information, generate information about which group the follow-up information corresponds to among a plurality of groups classified based on the size information of the first finding region and the second finding region included in the follow-up information.

The processor may be further configured to, when generating the diagnosis assistance information, generate quantitative information for each group related to a distribution of first finding region or second finding region to which the follow-up information corresponds for each of the plurality of groups.

The processor may be further configured to, when generating the quantitative information for each other, generate quantitative information for each group, including at least one of a number of pixels or voxels in finding regions corresponding to each of the plurality of groups, a number of finding regions corresponding to each section, or an area or volume of the finding regions corresponding to each section.

In the apparatus according to the embodiment of the present invention, the size information of the first finding region may be a length of a long axis of the first finding region, and the size information of the second finding region may be a length of a long axis of the second finding region; and the follow-up information may be a change between the length of the long axis of the first finding region and the length of the long axis of the second finding region.

In the apparatus according to the embodiment of the present invention, the size information of the first finding region may be a volume of the first finding region, and the size information of the second finding region may be a volume of the second finding region; and the follow-up information may be a change between the volume of the first finding region and the volume of the second finding region.

The processor may be further configured to, in accordance with at least one of the instructions, visualize information about which group the follow-up information corresponds to as the diagnosis assistance information.

### BRIEF DESCRIPTION OF THE DRAWINGS

FIG. 1 is an operational flowchart showing a method of generating diagnosis assistance information using medical images according to an embodiment of the present invention;

FIG. 2 is an operational flowchart showing part of the process of FIG. 1 in a method according to an embodiment of the present invention in detail;

FIG. 3 is an operational flowchart showing a method of generating and visualizing diagnosis assistance information using medical images according to another embodiment of the present invention;

FIG. 4 is a conceptual diagram showing the process of generating diagnosis assistance information for the evaluation of the degree of progression according to an embodiment of the present invention;

FIG. 5 is a conceptual diagram showing the process of generating diagnosis assistance information for the evaluation of the degree of progression according to another embodiment of the present invention;

FIG. 6 is a conceptual diagram showing an example of the diagnosis assistance information of FIG. 4;

FIG. 7 is a conceptual diagram showing an example of the diagnosis assistance information of FIG. 5; and

FIG. 8 is a conceptual diagram showing an example of the apparatus or generalized computing system for generating and visualizing diagnosis assistance information for the follow-up analysis of a specific disease.

### DETAILED DESCRIPTION

Other objects and features of the present invention in addition to the above-described objects will be apparent from the following description of embodiments to be given with reference to the accompanying drawings.

The embodiments of the present invention will be described in detail below with reference to the accompanying drawings. In the following description, when it is determined that a detailed description of a known component or function may unnecessarily make the gist of the present invention obscure, it will be omitted.

Relational terms such as first, second, A, B, and the like may be used for describing various elements, but the elements should not be limited by the terms. These terms are only used to distinguish one element from another. For example, a first component may be named a second component without departing from the scope of the present disclosure, and the second component may also be similarly named the first component. The term “and/or” means any one or a combination of a plurality of related and described items.

In embodiments of the present disclosure, “at least one of A and B” may mean “at least one of A or B” or “at least one of combinations of one or more of A and B.” Additionally, in embodiments of the present disclosure, “one or more of A and B” may mean “one or more of A or B” or “one or more of combinations of one or more of A and B.”

When it is mentioned that a certain component is “coupled with” or “connected with” another component, it should be understood that the certain component is directly “coupled with” or “connected with” to the other component or a further component may be disposed therebetween. In contrast, when it is mentioned that a certain component is “directly coupled with” or “directly connected with” another component, it will be understood that a further component is not disposed therebetween.

The terms used in the present disclosure are only used to describe specific exemplary embodiments, and are not intended to limit the present disclosure. The singular expression includes the plural expression unless the context clearly dictates otherwise. In the present disclosure, terms such as ‘comprise’ or ‘have’ are intended to designate that a feature, number, step, operation, component, part, or combination thereof described in the specification exists, but it should be understood that the terms do not preclude existence or addition of one or more features, numbers, steps, operations, components, parts, or combinations thereof.

Unless otherwise defined, all terms (including technical and scientific terms) used herein have the same meaning as commonly understood by one of ordinary skill in the art to which this disclosure belongs. Terms that are generally used and have been in dictionaries should be construed as having meanings matched with contextual meanings in the art. In this description, unless defined clearly, terms are not necessarily construed as having formal meanings.

Meanwhile, even if a technology is known prior to the filing date of the present disclosure, it may be included as part of the configuration of the present disclosure when necessary, and will be described herein without obscuring the spirit of the present disclosure. However, in describing the configuration of the present disclosure, a detailed description on matters that can be clearly understood by those skilled in the art as a known technology prior to the filing date of the present disclosure may obscure the purpose of the present disclosure, so excessively detailed description on the known technology will be omitted.

For example, technologies known before the application of the present invention may be used for a technology for the detection, segmentation, and classification of a specific organ of the human body and a sub-region of an organ by processing medical images, a technology for generating quantified information by measuring a segmented organ or finding region, etc. At least some of these known technologies may be applied as elemental technologies required for practicing the present invention.

In related prior literature, artificial neural networks are used to detect and classify lesion candidates and generate findings. Each of the findings includes diagnosis assistance information, and the diagnosis assistance information includes quantitative measurements such as the probability that each finding is actually a lesion, the confidence of the finding, the degree of malignancy, and the size and volume of lesion candidates corresponding to the finding.

In medical image diagnosis assistance using artificial neural networks, each finding needs to be quantified in the form of probability or confidence and included as diagnosis assistance information, and all findings may not be provided to a user. Accordingly, in general, findings are filtered by applying a specific threshold thereto, and only the findings obtained through the filtering are provided to the user. In a workflow in which a user, who is a radiologist, reads medical images and then generates clinical findings and a clinician analyzes the findings and then generates diagnosis results, an artificial neural network or automation program may assist in at least part of the reading process and finding generation process of the radiologist and the diagnosis process of the clinician.

However, the purpose of the present invention is not to claim rights to these known technologies, and the content of the known technologies may be included as part of the present invention within the range that does not depart from the spirit of the present invention.

Embodiments of the present invention will be described in detail below with reference to the accompanying drawings. In order to facilitate overall understanding in the following description of the present invention, the same reference numerals will be used for the same components throughout the drawings, and redundant descriptions thereof will be omitted.

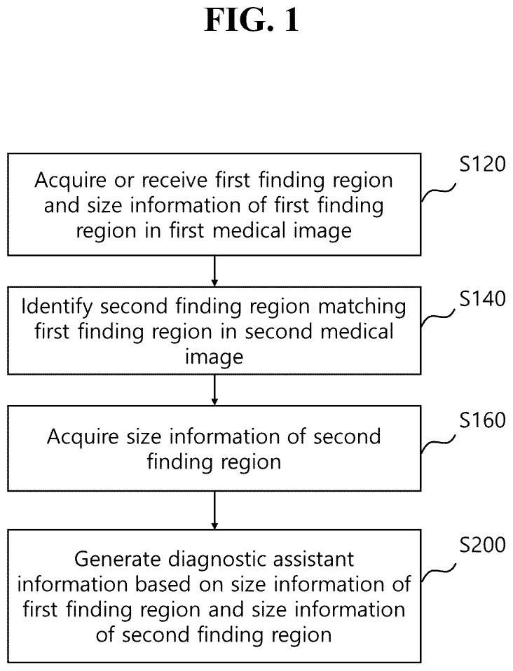

FIG. 1 is an operational flowchart showing a method of generating diagnosis assistance information using medical images according to an embodiment of the present invention.

Referring to FIG. 1, the method of generating diagnosis assistance information using medical images according to an embodiment of the present invention includes step S120 of obtaining a first finding region in a first medical image and the size information of the first finding region; step S140 of identifying a second fining region matching the first finding region in a second medical image obtained after the first medical image; step 160 of obtaining the size information of the second finding region; and step S200 of generating diagnosis assistance information based on the size information of the first finding region and the second finding region.

The finding region may be obtained through thresholding, detection, segmentation, and/or classification in the medical image.

The finding region may be a lesion or tumor, or may refer to an anatomical structure having a predetermined special shape or structure. Furthermore, the finding region may refer to an anatomical region identified through thresholding, detection, segmentation, and/or classification according to special conditions.

In the method of generating diagnosis assistance information using medical images according to an embodiment of the present invention, the first finding region may be a region whose intensity is lower than a first threshold in the first medical image, and the second finding region may be a region whose intensity is lower than the first threshold in the second medical image.

In the method of generating diagnosis assistance information using medical images according to an embodiment of the present invention, the first finding region may be a finding region related to the respiratory function of the lung within the first medical image, and the second finding region may be a finding region related to the respiratory function of the lung within the second medical image.

For example, when the intensity value of a region in a CT image is −950 HU (Hounsfield units), the region is sometimes referred to as a low attenuation area (LAA), and may generally be understood as a finding region corresponding to emphysema. In this case, −950 HU is merely a suggested value, and the spirit of the present invention is not limited thereby.

In an alternative embodiment of the present invention, a functional air trapping region may be obtained as a finding region.

An emphysema region or fAT region may be understood as the medical finding region related to the respiratory function of the lung.

An example of a medical finding region related to pulmonary disease may include a lung nodule region.

An alternative example of the medical finding region related to pulmonary disease may include a lung parenchyma region. Alternatively, an alternative example of the medical finding region related to pulmonary disease may include a lung texture region. Such lung texture regions may be classified into normal, ground-glass opacity (GGO), reticular opacity, linear opacity, nodular opacity, honeycombing, and consolidation.

The medical image may be a CT image, a magnetic resonance (MR) image, an X-ray image, or the like, and the finding region may include a medical finding region that can be obtained from a known modality.

Alternative examples of a finding region that can be extracted by considering intensity and shape in a medical image may include fat, blood, thrombus, etc.

The finding region may be obtained through detection, segmentation, or classification based on the results of thresholding of intensity values.

For example, the distribution of emphysema may be used as an index of the symptoms of COPD, and aerated lung parenchyma/ventilated functional tissue may generally be considered fibrous tissue that normally performs lung function.

Ground-glass opacity (GGO) refers to local nodular pulmonary infiltration. Generally, GGO may be defined as a nodule in which the boundaries of the bronchial tubes or blood vessels are defined well. Diffuse alveolar damage (DAD) and GGO are generally treated as opacity (OP). The distribution of OP may be considered an index of the symptoms of pneumonia.

Brightness value sections are set for each of fatty tissue (FAT), lymphatic tissue/lymphedema, leaky/transudate fluids, and exudate fluids, and a small region corresponding to each of the brightness value sections may be visualized through thresholding.

Furthermore, in addition to pneumonia and COPD, brightness value sections are set for a lung consolidation area, and the lung consolidation area may be separately subjected to thresholding. Lung consolidation generally refers to a condition in which liquid or cells replace the air in the alveoli, causing the lung to harden. In connection with lung consolidation, on an X-ray or CT image, lung OP increases relatively uniformly, there is little change in lung volume, and lung consolidation may be seen on an air bronchogram. Lung consolidation may also refer to a case where OP increases on a CT image to the extent that pulmonary blood vessels within a lesion cannot be identified.

In the method of generating diagnosis assistance information using medical images according to an embodiment of the present invention, the size information of the first finding region may be the length of the long axis of the first finding region, and the size information of the second finding region may be the length of the long axis of the second finding region. In this case, the follow-up information may be a change between the length of the long axis of the first finding region and the length of the long axis of the second finding region.

In the method of generating diagnosis assistance information using medical images according to an embodiment of the present invention, the size information of the first finding region may be the volume of the first finding region, and the size information of the second finding region may be the volume of the second finding region. In this case, the follow-up information may be a change between the volume of the first finding region and the volume of the second finding region.

The size information of the finding region may include the length of the long axis, the area/volume, the length of the perimeter, the surface area, the cross-sectional area, or the product of the long axis and the short axis. The size information of the first finding region and the second finding region that match each other may be calculated using the same method.

For convenience of description, the length of the long axis may be understood as referring to various measurement methods that can be used for the quantitative analysis of a specific finding region in a comprehensive manner. The quantitative measurement methods for a finding region include methods of measuring, e.g., the length of the long axis, the length of the short axis, the effective diameter, and/or the average length.

A new medical finding region or new size information is not necessarily required to implement the present invention. The configurational feature of the present invention is to match a finding region, known in related art, within the first medical image, which is a baseline image, and the second medical image, which is a follow-up image, by using pixel-to-pixel registration or matching and then generate or obtain clinically meaningful follow-up information by using a change in size information between matching finding regions obtained by a known method.

An embodiment of the present invention may propose a representation format for generating changes in size information between matching finding regions as clinically meaningful follow-up information. Furthermore, an embodiment of the present invention may obtain a means of quantitative evaluation of the degree of progression of a specific disease by quantitatively classifying a combination of the pieces of size information of matching finding regions or a change in size information between the matching finding regions.

An embodiment of the present invention may provide a means for visualizing the means of quantitative evaluation of the degree of progression of a specific disease obtained by quantitatively classifying a combination of the pieces of size information of matching finding regions or a change in size information between matching finding regions.

In an embodiment of the present invention, the pieces of size information of matching finding regions may be represented by an ordered pair such as (the size information of the first finding region, the size information of the second finding region). In an alternative embodiment of the present invention, the change in size information between finding regions may be represented by the difference or ratio in size information.

In an embodiment of the present invention, the pieces of size information of finding regions may be represented by values, or may be represented by size sections or groups classified by the values of the size information.

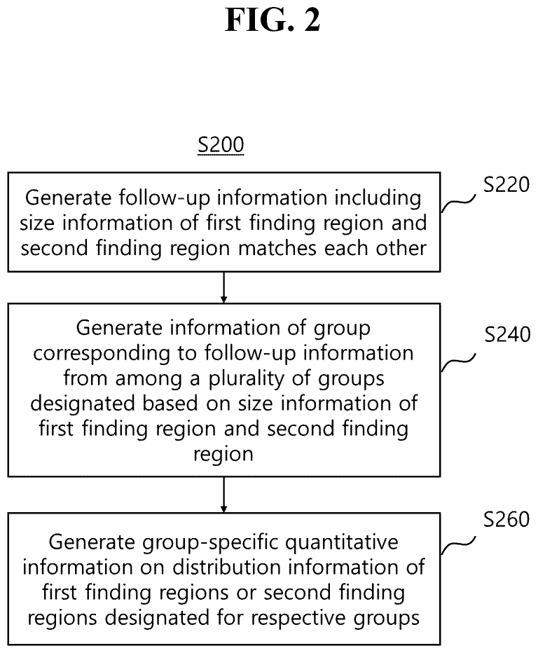

FIG. 2 is an operational flowchart showing part of the process of FIG. 1 in a method according to an embodiment of the present invention in detail.

Referring to FIG. 2, step S200 of generating diagnosis assistance information may include step S220 of generating follow-up information including the size information of the first finding region and the second finding region that match each other.

Step S200 of generating diagnosis assistance information may further include step S240 of generating information about which group the follow-up information corresponds to among a plurality of groups classified based on the size information of the first finding region and the second finding region included in the follow-up information.

Step S200 of generating diagnosis assistance information may further include step S260 of generating quantitative information for each group related to the distribution of at least one of the first finding region and/or the second finding region to which the follow-up information corresponds for each of the plurality of groups.

In step S260 of generating quantitative information for each group, there may be generated quantitative information for each group, including the number of pixels or voxels in finding regions corresponding to each of a plurality of groups, the number of finding regions corresponding to each section, or the area or volume of the finding regions corresponding to each section.

In this case, the quantitative information for each group may refer to a section of the size information of the first finding region, and, for groups classified for each section of the size information of the second finding region, the distribution of finding regions corresponding to each group, or the distribution of pixels or voxels corresponding to each group.

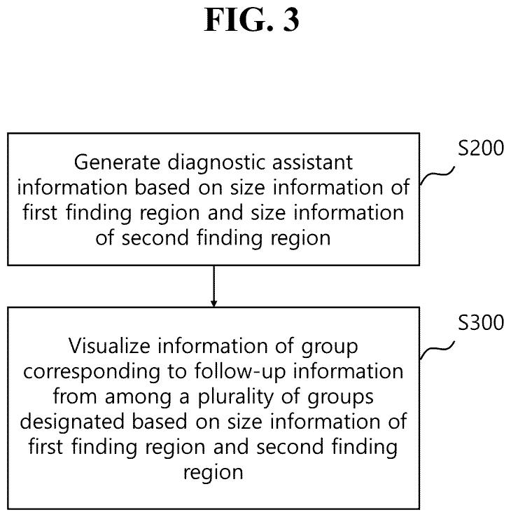

FIG. 3 is an operational flowchart showing a method of generating and visualizing diagnosis assistance information using medical images according to another embodiment of the present invention.

Referring to FIG. 3, the method of generating diagnosis assistance information using medical images according to an embodiment of the present invention may further include step S300 of visualizing information about which group the follow-up information corresponds to, which is the step of visualizing the diagnosis assistance information obtained in step S200.

Referring again to FIG. 1, in an alternative embodiment of the present invention, step S200 of generating diagnosis assistance information may further include the step of generating diagnosis assistance information, including information about which section the follow-up information corresponds to among a plurality of sections classified by applying a plurality of thresholds to the change between the size information of the first finding region and the size information of the second finding region included in the follow-up information.

Step S200 of generating diagnosis assistance information may further include the step of generating diagnosis assistance information, including quantitative information for each section related to the distribution of first finding region or second finding region to which the follow-up information corresponds for each of the plurality of sections.

In this case, regardless of the absolute value of the size of the first finding region in the baseline image and the size of the second finding region in the follow-up image, only the change amount/change rate may be classified as a threshold and a section or group may be set.

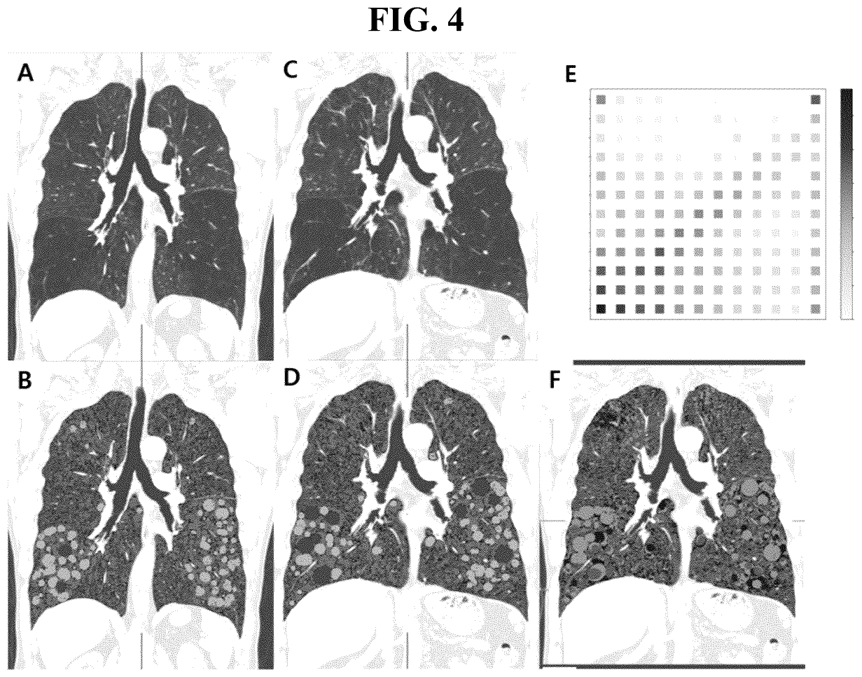

FIG. 4 is a conceptual diagram showing the process of generating diagnosis assistance information for the evaluation of the degree of progression according to an embodiment of the present invention.

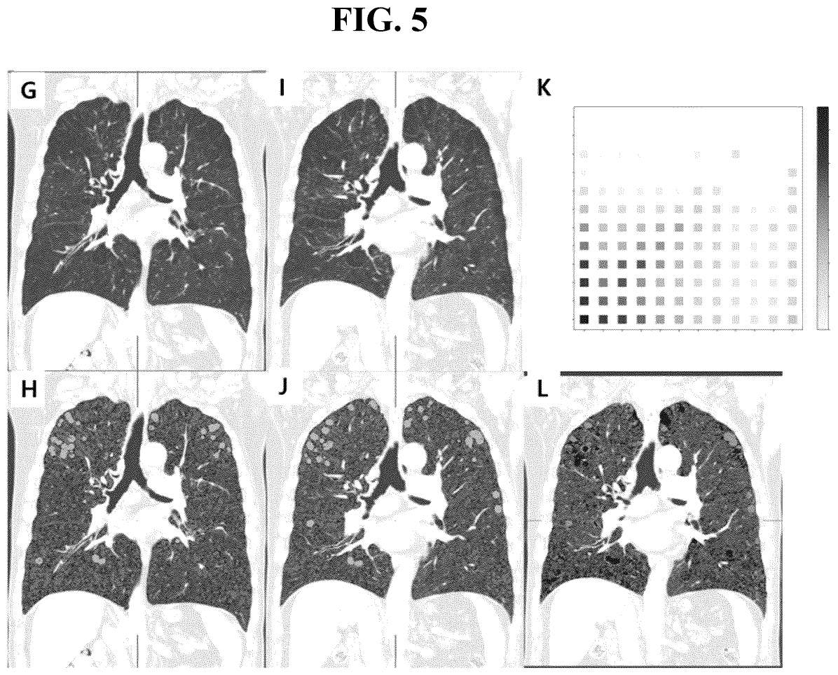

FIG. 5 is a conceptual diagram showing the process of generating diagnosis assistance information for the evaluation of the degree of progression according to another embodiment of the present invention.

FIG. 4 shows the results of analysis of baseline and follow-up images of a first COPD patient, and FIG. 5 shows the results of analysis of baseline and follow-up images of a second COPD patient.

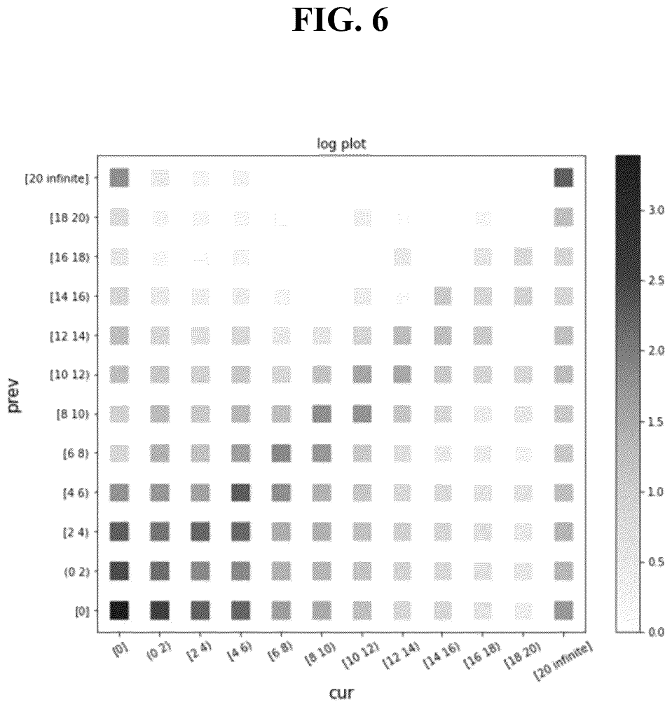

FIG. 6 is a conceptual diagram showing an example of the diagnosis assistance information of FIG. 4.

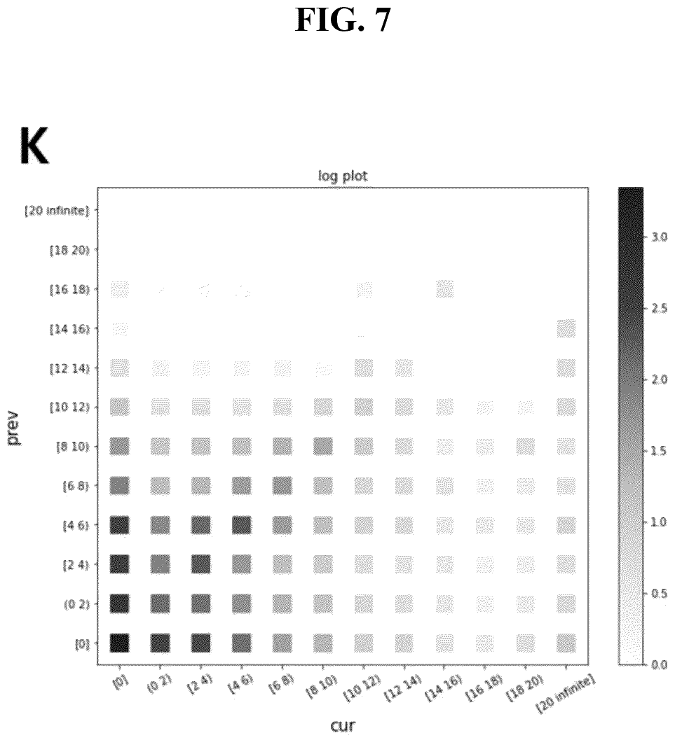

FIG. 7 is a conceptual diagram showing an example of the diagnosis assistance information of FIG. 5.

Referring to FIGS. 4 to 7 together, image A may be the baseline chest CT image of the first patient. Image B may be a mask for a finding region for emphysema in the baseline CT image of the first patient. In this case, finding regions for emphysema in image B may be represented using different visual elements (different colors, brightness levels, or the like) based on size. For example, the finding regions for emphysema may be classified into a plurality of groups based on a plurality of thresholds of size values, and different visual elements may be allocated to respective groups to which individual finding regions belong and represented by masks for the respective groups. For example, as a finding region for emphysema belongs to a group having a larger size value, it may be represented by a brighter or darker mask on image B. In this specification, a mask that is classified differently depending on the size value thereof may be referred to as a size mask.

Image C may be the follow-up chest CT image of the first patient. Image D may be a size mask for finding regions for emphysema in the follow-up CT image of the first patient.

Image E may be a graph showing changes in the sizes of finding regions for emphysema derived from the follow-up information of the first patient. In this case, referring to FIG. 6, the horizontal axis may represent the size section of the second finding region in the follow-up image [cur], and the vertical axis may represent the size section of the first finding region in the baseline image [prev].

In this case, when the size of the finding region is 0 mm, it may refer to a normal region.

The size information of the first finding region and the size information of the second finding region may be classified as a [0, 2] mm section and a [2, 4] mm section, respectively. Furthermore, the number of pixels/voxels of the second finding region may be accumulated for each group determined according to the section to which the size information of the first finding region belongs and the section to which the size information of the second finding region belongs. As the number of pixels/voxels accumulated for each group increases, representation may be performed using a darker color. When the size of the first finding region corresponds to a [0, 2] section and the size of the second finding region corresponds to a [2, 4] section, the number of pixels/voxels of the second finding region may be accumulated for a [0, 2]-[2, 4] group.

In graph E, the color of a [0]-[0] group may denote the normal number of pixels/voxels, i.e., an effective volume, in both the baseline and follow-up images. In graph E, groups on a diagonal line connecting the [0]-[0] group, and [0 2]-[0 2], . . . , and [20 infinite]-[20 infinite] sections may represent the level at which the progression of disease is maintained. In graph E, it may be appreciated that the groups in the lower right part of the diagonal line denote the level at which the progression of disease becomes worse and the groups in the upper left side of the diagonal line denote the level at which the patient recovers from the disease.

Image F may be an image that visually represents changes in the size of emphysema in the chest CT image of the first patient. In this case, in the follow-up image, the finding regions whose sizes increased, the finding regions whose sizes are substantially the same (which refer to cases where changes in size are lower than a threshold), and the finding regions whose sizes decreased may be visualized using different visual elements (colors, brightness levels, or the like).

In an alternative embodiment, in image F, individual finding regions may be visualized using different visual elements so that as the change in size increases, it is emphasized more.

In image F, there may be visualized diagnosis assistance information that assists in an intuitive understanding of where the finding regions whose sizes increased are distributed and where the finding regions which have large changes in size are distributed.

In other words, it may be appreciated that image E is a graph visualizing changes in the sizes of the finding regions within the overall lung area and image F is a means for visualizing the distribution of finding regions whose sizes increased or which have significant changes in size.

In an alternative embodiment, image E may be calculated and provided differently for each sub-region of the lung area. For example, image E may be provided for each of the core region, rind region and peri region of the lung. Alternatively, image E may be provided for each of the five lung lobes of the lung. In this case, the five lung lobes are the right upper lobe (RUL), the right middle lobe (RML), the right lower lobe (RLL), the left upper lobe (LUL), and the left lower lobe (LLL).

Image G may be the baseline chest CT image of the second patient. Image H may be a size mask for finding regions for emphysema in the baseline CT image of the second patient. Image I may be the follow-up chest CT image of the second patient. Image J may be a size mask for finding regions for emphysema in the follow-up CT image of the second patient.

Image K may be a graph showing changes in the sizes of the finding regions for emphysema derived from the follow-up information of the second patient.

Image L may be an image visually representing changes in the size of emphysema in the chest CT image of the second patient.

The first patient in FIGS. 4 and 6 corresponds to a case where LAA % (the area/volume ratio of the LAA area) increased from 46% to 52%, and the second patient in FIGS. 5 and 7 corresponds to a case where LAA % remained at 39%.

In the embodiments of FIGS. 4 to 7, there are shown visualization means for providing intuitive understanding to medical professionals. In an alternative embodiment of the present invention, the area/volume % distribution of each of the groups classified by ordered pairs for each size section of finding regions may be provided as a table.

Visualization tools, e.g., a graph such as images E and K, and a size change mask overlaid on a medical image such as images F and L, and a table containing statistical information may be provided as means for quantitatively evaluating the degree of progression of a specific disease based on follow-up information in the form of a report.

Although there have been disclosed the embodiments that provide the means for quantitatively evaluating the degree of progression of COPD mainly through finding regions for emphysema in FIGS. 1 to 7, the technical spirit of the present invention is not limited to the specific embodiments.

In an alternative embodiment of the present invention, there may be obtained and visualized the quantitative distribution of changes in the sizes of lung texture finding regions used to evaluate the degree of progression of interstitial lung disease (ILD), also called pulmonary fibrosis. Alternatively, to evaluate the degree of progression of a specific disease, there may be obtained and visualized the quantitative distribution of changes in the sizes using follow-up information for the size information of known finding regions.

To perform the processes of FIGS. 1 to 7, for example, voxel-to-voxel analysis results may be obtained between baseline and follow-up images for the overall lung area regardless of whether there is a lesion.

The process of obtaining size-based statistical information for first finding regions and the process of obtaining size-based statistical information for second finding regions may be performed independently of each other. In this case, the size-based statistical information may include the sizes of the finding regions (=the number of pixels/voxels corresponding to the finding regions), and the distribution based on the sizes of the finding regions (=the cumulative number of pixels/voxels of the finding regions for each section/group classified according to size).

In an embodiment of the present invention, a plurality of adjacent first finding regions in a baseline image may grow and merge and be classified as one second finding region in a follow-up image. In this case, the plurality of first finding regions may match the one second finding region. Conversely, one first finding region in a baseline image may become smaller and separated and be classified as two or more adjacent second finding regions in a follow-up image. In this case, the one first finding region may match the plurality of second finding regions.

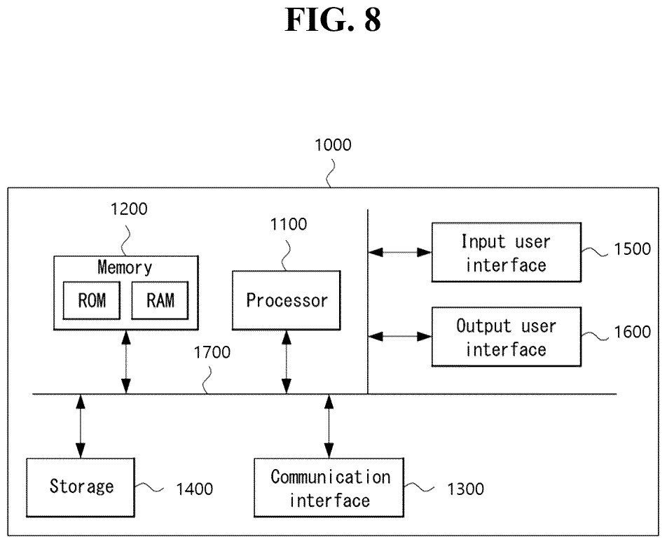

FIG. 8 is a conceptual diagram showing an example of the apparatus for generating and visualizing diagnosis assistance information for the follow-up analysis of a specific disease using generalized medical image processing, analysis and visualization, diagnosis assistance, and/or medical images, or the apparatus or computing system for assisting in the diagnosis of a specific disease through follow-up analysis that are capable of performing at least part of the processes of FIGS. 1 to 7.

At least part of the method of generating and visualizing diagnosis assistance information using medical image processing, analysis and visualization, diagnosis assistance, and/or medical images and the method of assisting in the diagnosis of a specific disease through follow-up analysis may be performed by the computing system 1000 of FIG. 8.

Referring to FIG. 8, the computing system 1000 according to an embodiment of the present invention may include a processor 1100, memory 1200, a communication interface 1300, storage 1400, an input user interface 1500, an output user interface 1600, and a bus 1700.

The computing system 1000 according to an embodiment of the present invention may include the at least one processor 1100, and the memory 1200 configured to store instructions causing the at least one processor 1100 to perform at least one step. At least part of the method according to an embodiment of the present invention may be performed in such a manner that the at least one processor 1100 loads instructions from the memory 1200 and executes them.

The processor 1100 may refer to a central processing unit (CPU), a graphics processing unit (GPU), or a dedicated processor configured such that the methods according to the embodiments of the present invention are performed thereon.

Each of the memory 1200 and the storage 1400 may include at least one of a volatile storage medium and a non-volatile storage medium. For example, the memory 1200 may include at least one of read-only memory (ROM) and random access memory (RAM).

The computing system 1000 may further include the communication interface 1300 configured to perform communication over a wireless network.

The computing system 1000 may further include the storage 1400, the input user interface 1500, and the output user interface 1600.

The individual components included in the computing system 1000 may be connected through the bus 1700 and communicate with each other.

Examples of the computing system 1000 according to the present invention may include a communication-enabled desktop computer, laptop computer, notebook computer, smartphone, tablet personal computer (PC), mobile phone, smart watch, smart glass, e-book reader, portable multimedia player (PMP), portable game console, car navigation device, digital camera, digital multimedia broadcasting (DMB) player, digital audio recorder, digital audio player, digital video recorder, digital video player, and personal digital assistant (PDA), etc.

An apparatus for analyzing medical images to assist in diagnosing spinal disease according to an embodiment of the present invention may include memory 1200 configured to store one or more instructions, and a processor 1100 configured to load and execute at least one instruction from the memory.

In accordance with at least one instruction, the processor 1100 may obtain a first finding region and the size information of the first finding region within a first medical image, may identify a second finding region matching the first finding region in a second medical image obtained after the first medical image, may obtain the size information of the second finding region, and may generate diagnosis assistance information based on the size information of the first finding region and the size information of the second finding region.

When generating the diagnosis assistance information, the processor 1100 may generate follow-up information including the size information of the first finding region and the second finding region that match each other.

When generating the diagnosis assistance information, the processor 1100 may determine information about which group the follow-up information corresponds to among a plurality of groups classified based on the size information of the first finding region and the second finding region included in the follow-up information

When generating the diagnosis assistance information, the processor 1100 may generate quantitative information for each group related to the distribution of first finding regions or second finding regions to which the follow-up information corresponds for each of a plurality of groups.

When generating the quantitative information for each group, the processor 1100 may generate quantitative information for each group, including the number of pixels or voxels in finding regions corresponding to each of the plurality of groups, the number of finding regions corresponding to each section, or the area or volume of the finding regions corresponding to each section.

In the apparatus for generating diagnosis assistance information using medical images according to an embodiment of the present invention, the size information of the first finding region may be the length of the long axis of the first finding region, and the size information of the second finding region may be the length of the long axis of the second finding region. In this case, the follow-up information may be a change between the length of the long axis of the first finding region and the length of the long axis of the second finding region.

In the apparatus for generating diagnosis assistance information using medical images according to an embodiment of the present invention, the size information of the first finding region may be the volume of the first finding region, and the size information of the second finding region may be the volume of the second finding region. In this case, the follow-up information may be a change between the volume of the first finding region and the volume of the second finding region.

In accordance with at least one instruction, the processor 1100 may visualize information about which group the follow-up information corresponds to as diagnosis assistance information.

According to an embodiment of the present invention, a consistent index for quantitative analysis may be provided using registration between the latest image and the previous image.

According to an embodiment of the present invention, there may be provided an evaluation index, the visualization of the index, and evaluation criteria for the quantitative evaluation of the degree of progression of COPD or emphysema.

According to an embodiment of the present invention, an index capable of quantitatively evaluating the degree of progression of a specific disease may be generated based on changes in the size of a finding region over time by using the follow-up matching of finding regions obtained through thresholding, detection, segmentation, and/or classification in medical images.

In this case, criteria for determining a fining region, criteria for determining the size of the finding region, and/or criteria for evaluating a change in the size of the finding region may be determined depending on a specific disease. Furthermore, a quantification method for calculating the size of a finding region and/or a change in the size of the finding region may be selected from clinically known methods.

The operations of the method according to the exemplary embodiment of the present disclosure can be implemented as a computer readable program or code in a computer readable recording medium. The computer readable recording medium may include all kinds of recording apparatus for storing data which can be read by a computer system. Furthermore, the computer readable recording medium may store and execute programs or codes which can be distributed in computer systems connected through a network and read through computers in a distributed manner.

The computer readable recording medium may include a hardware apparatus which is specifically configured to store and execute a program command, such as a ROM, RAM or flash memory. The program command may include not only machine language codes created by a compiler, but also high-level language codes which can be executed by a computer using an interpreter.

Although some aspects of the present disclosure have been described in the context of the apparatus, the aspects may indicate the corresponding descriptions according to the method, and the blocks or apparatus may correspond to the steps of the method or the features of the steps. Similarly, the aspects described in the context of the method may be expressed as the features of the corresponding blocks or items or the corresponding apparatus. Some or all of the steps of the method may be executed by (or using) a hardware apparatus such as a microprocessor, a programmable computer or an electronic circuit. In some embodiments, one or more of the most important steps of the method may be executed by such an apparatus.

In some exemplary embodiments, a programmable logic device such as a field-programmable gate array may be used to perform some or all of functions of the methods described herein. In some exemplary embodiments, the field-programmable gate array may be operated with a microprocessor to perform one of the methods described herein. In general, the methods are preferably performed by a certain hardware device.

The description of the disclosure is merely exemplary in nature and, thus, variations that do not depart from the substance of the disclosure are intended to be within the scope of the disclosure. Such variations are not to be regarded as a departure from the spirit and scope of the disclosure. Thus, it will be understood by those of ordinary skill in the art that various changes in form and details may be made without departing from the spirit and scope as defined by the following claims.

What is claimed is:

1. A method of generating diagnosis assistance information using medical images, the method comprising:
obtaining a first finding region in a first medical image and size information of the first finding region;
identifying a second finding region matching the first finding region in a second medical image obtained after the first medical image;
obtaining size information of the second finding region; and
generating diagnosis assistance information based on the size information of the first finding region and the second finding region.

2. The method of claim 1, wherein the generating comprises generating follow-up information including the size information of the first finding region and the second finding region that match each other.

3. The method of claim 2, wherein the generating further comprises generating information about which group the follow-up information corresponds to among a plurality of groups classified based on the size information of the first finding region and the second finding region included in the follow-up information.

4. The method of claim 3, wherein the generating further comprises generating quantitative information for each group related to a distribution of the first finding region or the second finding region to which the follow-up information corresponds for each of the plurality of groups.

5. The method of claim 4, wherein the generating quantitative information for each other comprises generating quantitative information for each group, including at least one of a number of pixels or voxels in finding regions corresponding to each of the plurality of groups, a number of finding regions corresponding to each section, or an area or volume of the finding regions corresponding to each section.

6. The method of claim 2, wherein the size information of the first finding region is a length of a long axis of the first finding region, and the size information of the second finding region is a length of a long axis of the second finding region, and
wherein the follow-up information is a change between the length of the long axis of the first finding region and the length of the long axis of the second finding region.

7. The method of claim 2, wherein the size information of the first finding region is a volume of the first finding region, and the size information of the second finding region is a volume of the second finding region, and
wherein the follow-up information is a change between the volume of the first finding region and the volume of the second finding region.

8. The method of claim 3, further comprising:
visualizing information about which group the follow-up information corresponds to as the diagnosis assistance information.

9. The method of claim 2, wherein the generating further comprises generating diagnostic assistance information, including information about which section the follow-up information corresponds to among a plurality of sections classified by applying a plurality of thresholds to a change between the size information of the first finding region and the second finding region included in the follow-up information.

10. The method of claim 9, wherein the generating further comprises generating diagnosis assistance information, including quantitative information for each section related to a distribution of first finding region or second finding region to which the follow-up information corresponds for each of the plurality of sections.

11. The method of claim 1, wherein the first finding region is a region in the first medical image whose intensity is lower than a first threshold, and
wherein the second finding region is a region in the second medical image whose intensity is lower than the first threshold.

12. The method of claim 1, wherein the first finding region is a finding region related to a respiratory function of a lung in the first medical image, and
wherein the second finding region is a finding region related to the respiratory function of the lung in the second medical image.

13. An apparatus for generating diagnosis assistance information using medical images, the apparatus comprising:
memory configured to store one or more instructions; and
a processor configured to execute the one or more instructions,
wherein the processor is further configured to, in accordance with at least one of the instructions:
obtain a first finding region in a first medical image and size information of the first finding region;
identify a second finding region matching the first finding region in a second medical image obtained after the first medical image;
obtain size information of the second finding region; and
generate diagnosis assistance information based on the size information of the first finding region and the second finding region.

14. The apparatus of claim 13, wherein the processor is further configured to, when generating the diagnosis assistance information, generate follow-up information including the size information of the first finding region and the second finding region that match each other.

15. The apparatus of claim 14, wherein the processor is further configured to, when generating the diagnosis assistance information, generate information about which group the follow-up information corresponds to among a plurality of groups classified based on the size information of the first finding region and the second finding region included in the follow-up information.

16. The apparatus of claim 15, wherein the processor is further configured to, when generating the diagnosis assistance information, generate quantitative information for each group related to a distribution of first finding region or second finding region to which the follow-up information corresponds for each of the plurality of groups.

17. The apparatus of claim 16, wherein the processor is further configured to, when generating the quantitative information for each other, generate quantitative information for each group, including at least one of a number of pixels or voxels in finding regions corresponding to each of the plurality of groups, a number of finding regions corresponding to each section, or an area or volume of the finding regions corresponding to each section.

18. The apparatus of claim 14, wherein the size information of the first finding region is a length of a long axis of the first finding region, and the size information of the second finding region is a length of a long axis of the second finding region, and
wherein the follow-up information is a change between the length of the long axis of the first finding region and the length of the long axis of the second finding region.

19. The apparatus of claim 14, wherein the size information of the first finding region is a volume of the first finding region, and the size information of the second finding region is a volume of the second finding region, and
wherein the follow-up information is a change between the volume of the first finding region and the volume of the second finding region.

20. The apparatus of claim 15, wherein the processor is further configured to, in accordance with at least one of the instructions, visualize information about which group the follow-up information corresponds to as the diagnosis assistance information.
